(19) United States
(12) Patent Application Publication
(10) Pub. No.: US 2019/0125177 A1
(43) Pub. Date: May 2, 2019

(54) INTEGRATED VIDEO LARYNGOSCOPE MOUNTED STYLET AND DEPLOYMENT SYSTEM FOR ENDOTRACHEAL TUBES AND DOUBLE-LUMEN ENDOBRONCHIAL TUBES

(71) Applicant: LEROY NICHELOUS SUTHERLAND, BERKELEY HEIGHTS, NJ (US)

(72) Inventor: LEROY NICHELOUS SUTHERLAND, BERKELEY HEIGHTS, NJ (US)

(21) Appl. No.: 15/963,947

(22) Filed: Apr. 26, 2018

Related U.S. Application Data
(60) Provisional application No. 62/490,204, filed on Apr. 26, 2017.

(51) Int. Cl. A61B 1/267; A61B 1/05; A61M 16/04

(52) U.S. Cl. CPC A61B 1/267; A61B 1/07; A61M 16/0488; A61B 1/05

(57) ABSTRACT

In one aspect of the present disclosure is an endotracheal intubation system for performing an endotracheal intubation, comprising: a laryngoscope including a laryngoscope blade, the laryngoscope including a channel having a plurality of recesses configured to releasably engage an ETT/DLT carrier/holder; an endotracheal tube releasably secured to the ETT/DLT carrier/holder by fixation means; and a coaxial stylet for the endotracheal tube, the coaxial stylet including inner and outer stylets and an inner stylet steering arm.

### CROSS REFERENCE TO RELATED APPLICATIONS

The present application claims the benefit of the filing date of U.S. Provisional Patent Application No. 62/490,204 filed Apr. 26, 2017, the disclosure of which is hereby incorporated herein by reference in its entirety.

### BACKGROUND OF THE DISCLOSURE

Endotracheal intubation is a critical means for maintaining the breathing function of a patient under general anesthesia. In most cases, to prevent the occurrence of hypoxia, medical professionals must complete the intubation by inserting an endotracheal tube into the patient's trachea in a very short period of time to provide oxygen there into promptly. Thus, it is vital that medical professionals perform the intubation efficiently.

However, due to the structural differences of the upper airways among patients of different ages or sizes, the patients' tracheas sometimes cannot be easily located. Thus, in practice, medical professionals always have auxiliary tools at hand to facilitate the intubation process. For example, an endotracheal tube stylet may be inserted into an endotracheal tube and bent to a specific curvature in advance; then a laryngoscope is adapted to depress the patient's tongue base so that a medical professional may put the endotracheal tube and the endotracheal tube stylet through the patient's mouth, where the endotracheal tube stylet is withdrawn when the endotracheal tube is inserted into the trachea.

Some of the difficulties the medical professional (hereinafter “user”) encounters include the restriction of view as the tube is inserted, variations in the anatomy of the patients, an uncomfortable and unnatural position for the anesthesiologist while holding the instrument and the necessity for rapid intubation.

For example, video laryngoscopes have been used to help facilitate the intubation of a patient. Video laryngoscopes typically contain a light guiding system, usually in the form of fiber optic cables, to bring light to the procedural area. Video laryngoscopes also typically contain an image guiding system, for example in the form of a rigid rod lens system, arranged in the blade of the laryngoscope, or in the form of an ordered, flexible fiber optic bundle. In these configurations, the image guiding system is utilized to transmit reflected light from the area ahead of the blade to a camera, which may be attached to the laryngoscope. Alternatively, it is known to affix an imager, which may comprise, for example, a Charge Couple Device (CCD), or Complementary Metal Oxide Semiconductor (CMOS) to the distal end of the laryngoscope blade. In this case, the image data may be transmitted to the camera affixed to the laryngoscope via electrical wires (or wirelessly) as digital image data.

With the advent of video laryngoscopes and cameras, instrumentation has been improved to the extent that it can enable viewing of the cords and larynx on a video screen thereby facilitating the intubation of the patient in an overall relatively quick and safe manner. Typically, however, though the equipment achieves a higher percentage of glottic opening view than standard direct laryngoscopy, passage of the endotracheal tube (“ETT”), is not universally easy. Often the combination of limited room for manipulation of the ETT, occurring while maintaining and watching a projected image that provides poor depth perception and obscuration of the target view by the advancing ETT results in longer intubation times.

While this configuration for a video laryngoscope does provide a user very useful information in the form of image data, a problem with current systems is the limited field of view during ETT deployment as the ETT blocks views of the vocal cords as it approaches the laryngeal inlet. For example, when the user is intubating the patient, the imaging device, typically positioned on the underside and distal end of the laryngoscope blade, only provides a view of the, roughly, two-thirds of the configuration of the anatomy (depending on the patient it could be more or less) with portions of the larynx not visible to the user in many instances. This is because the laryngoscope is only advanced into the throat of the patient far enough to lift the tongue and the epiglottis sufficiently to view the vocal cords and facilitate the introduction of the ETT, into the trachea not necessarily to get the best possible view.

A malleable aluminum stylet is an accessory used with the ETT (typically inserted into the tube) to provide the tube with additional rigidity for the intubation process. The anatomy of patients often requires that the tip of the ETT to have a sharper bend and be partially more rigid so as to introduce it through the vocal cords, which are located toward an anterior (at the 12 o'clock) position. The stylet, which may comprise a malleable aluminum rod covered with a plastic material (disposable) is slid inside the ETT and is used to increase the bend of the tip of the ETT and form the proper angulation for the particular patient. After intubation, the stylet is removed and the ETT remains in place.

### BRIEF SUMMARY OF THE DISCLOSURE

A reliable laryngoscopy view can usually be expected with a video laryngoscope, especially with blades of maximal angulation. However, passage of the ETT/DLT is not infrequently challenging. This is due to non-alignment of the axes of the oropharynx with the laryngotracheal airway. Indeed, best alignment of axes is usually achieved when there is a less than optimal laryngoscopic view, a nuanced approach often achieved only after extensive experience is acquired. Additionally, narrow visual fields result in obscuration of the laryngeal structures as the endotracheal tube is advanced. Blind advancement of existing stylet systems often results in over exuberant distal deployment of the stylet resulting in possible distal airway (usually minor) trauma but more importantly, in the extreme, stimulation of the very sensitive carinal cough reflex in more lightly anesthetized patients (not an uncommon clinical scenario when awakening the patient is a prudent decision encouraged in the ASA difficult airway algorithm if intubation is proving difficult to achieve). Induction of the cough reflex, airway trauma, bleeding, potential bronchospasm, laryngospasm and increased procedural difficulty in a reactive patient further complicates and endangers the patient as chances of successful intubation are reduced. The ability to correct for these deficiencies would be of particular benefit to (i) first responders in the field often operating in less than optimal conditions, (ii) where more expensive equipment and steep learning curves render other techniques impractical and (iii) airway management trainees when acquiring the best possible view is the only significant learning curve requirement.

In one aspect of the present disclosure is an endotracheal intubation system for performing an endotracheal intubation, comprising: a laryngoscope including a laryngoscope blade, the laryngoscope including a channel having a plurality of recesses configured to releasably engage an ETT/DLT carrier/holder; an endotracheal tube releasably secured to the ETT/DLT carrier/holder by fixation means; and a coaxial stylet for the endotracheal tube, the coaxial stylet including inner and outer stylets and an inner stylet steering arm. In some embodiments, the fixation means comprise a connector cap and cuff holder wires. In some embodiments, the carrier /holder interlock mechanism is superseded by a “direct coaxial stylet conduit,” a machined cylindrical channel with a linear inner stylet release slot, variably embedded in the substance of the blade and of a variable length determined by the need for incidental emergency use as opposed to routine, every intubation attempt use. In some embodiments, the system further comprises a transition free conical cuff. In some embodiments, the coaxial stylet is configured to pass through a hollow tubular segment of the transition free conical cuff. In some embodiments, the coaxial stylet comprises at least one means for restricting advancement of the inner stylet. In some embodiments, the at least one means for restricting advancement of the inner stylet is a stop limit disc coupled to an end of the outer stylet. In some embodiments, the at least one means for restricting advancement of the inner stylet is a stop limit ring positioned around an outer surface of the outer stylet. In some embodiments, the ETT/DLT carrier/holder is reliably engaged to the recesses of the laryngoscope by a plurality of interlock tabs. In some embodiments, the inner stylet comprises an arcuate or curved navigational end, the arcuate or curved navigational end opposite the inner stylet steering arm. In some embodiments, the inner stylet comprises an atraumatic spherical tip.

Integral to the system is the idea of an interlock between the blade and the achieved view with the delivery of the ETT. The variability of tolerable blade modifications results in the presentation of multiple potential approaches to optimize solutions given the wide range of existing blade designs

### BRIEF SUMMARY OF THE FIGURES

FIG. 1A provides a perspective view of a modified video laryngoscope blade in accordance with some embodiments.

FIG. 1B provides a perspective view of a short segment type of “direct coaxial stylet conduit” blade modification (segment shown not embedded) in accordance with some embodiments.

FIG. 1C illustrates a cut-away side view of a modified blade showing an interlock channel in accordance with some embodiments.

FIG. 1D sets forth a front view of a modified blade showing an interlock channel in accordance with some embodiments.

FIG. 1E illustrates a cross-section of an interlock channel showing varying levels of embedding into the blade in accordance with some embodiments.

FIG. 1F illustrates a cross-sectional view of an interlock channel showing varying levels of embedding into the blade in accordance with some embodiments.

FIG. 1G illustrates a cross-section of an interlock channel showing varying levels of embedding into the blade in accordance with some embodiments.

FIG. 2A provides a perspective view of a ELT/DLT carrier/holder in accordance with some embodiments.

FIG. 2B provides a perspective view of an ETT or DLT loaded onto a ELT/DLT carrier/holder in accordance with some embodiments. The figure illustrates a coaxial slide segment of wire in a hollow tubular extension of the cap.

FIG. 2C illustrates a cross-sectional view of the connector of the ELT/DLT carrier /holder in accordance with some embodiments.

FIG. 2D provides an exploded view of an interlock tab of the ELT/DLT carrier/ holder of FIG. 2A.

FIG. 3A provides a perspective view of a transition free conical cuff, the transition free conical cuff shown with an inflated cuff in accordance with some embodiments. The figure illustrates a cuff on a hollow tubular segment, a proximal malleable low profile reinforced structural element, and a pilot cuff and clip.

FIG. 3B provides a perspective view of a coaxial stylet in accordance with some embodiments. The figure illustrates an atraumatic spherical tip, a curved navigational tip, a distal inner stylet, an outer stylet, a stop limiting ring, a stop limiter for the outer stylet, and a proximal inner stylet steering arm.

FIG. 4A provides a perspective view of a fully loaded system comprising a carrier holder interlock device according to the present disclosure in accordance with some embodiments. The fully loaded system illustrates a proximal portion of an inner stylet with an integrated steering arm (400), a proximal portion of an outer stylet with integrated stop limiter (401), a carrier/holder proximal ETT/DLT connector (402), a conical cuff structural element exiting slot in the carrier/ holder connector (403), a conical cuff pilot balloon (404), a funnel-shaped, tapered slot entrance to the interlock groove of the blade (405), an interlock tab installed in a modified video laryngoscope blade (406), an ETT pilot balloon (420), a distal portion of the inner stylet (416), a distal portion of the outer stylet (417), an inflated conical cuff creating a transition free interface (418), and an inflated ETT cuff secured in a carrier/holder (419).

FIG. 4B illustrates a cross-sectional view of the fully loaded device of FIG. 4A. The view further illustrates an inflated ETT cuff with bulge at a wire free segment (407), a reinforced zone of a transition free conical cuff with pilot tube lumen (408), an inflated conical cuff opposed to the ETT (409), an ETT wall 410, a ETT/DLT carrier/holder wire (411), an interlock tab of the ETT/DLT carrier/holder (413), an outer stylet (414), and an inner stylet (415).

FIG. 4C provides a cross-sectional view of the system of FIG. 4A. The view further illustrates a transition free biconical cuff wall (41), an outer stylet (42), an inner stylet (43), an ETT holder with retained inflated ETT cuff (44), a bulging inflated ETT cuff (45), a wall of an ETT (46), an inflated biconical cuff (47), and a pilot tubing of a biconical cuff (48).

FIG. 5A sets forth an alternate pediatric stylet design for use with the system of the present disclosure. The figure further illustrates low friction contact surface reducing ridges (501) and a size specific solid integrated transition free zone (502).

FIG. 5B sets forth direct coaxial stylet conduit outer stylet retention segment enlargement options of an alternate embodiment.

FIG. 5C sets forth another alternative pediatric stylet design for use with the system of the present disclosure. The figure illustrates a stylet with an integrated cuff for small ETT/DLTs transition free zone (503).

FIG. 6A illustrates an interlock stylet in accordance with some embodiments. A virtual interlock is achieved when a rigid free-standing stylet, that has a complementary curvature to the blade with which it is used, is passed on an arc parallel to the blades curvature. In combination with the benefits of the coaxial stylet this should improve success rates.

FIG. 6B illustrates an interlock stylet in accordance with some embodiments, and specifically one showing a typical curve.

FIG. 7A provides a flowchart illustrating a method of assembling a system comprising a carrier holder interlock device, and the use of the system during a medical procedure in accordance with some embodiments.

FIG. 7B provides a flowchart illustrating a method of assembling the system of the present disclosure, including the utilization of a direct coaxial stylet conduit, and the use of the system during a medical procedure in accordance with some embodiments.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

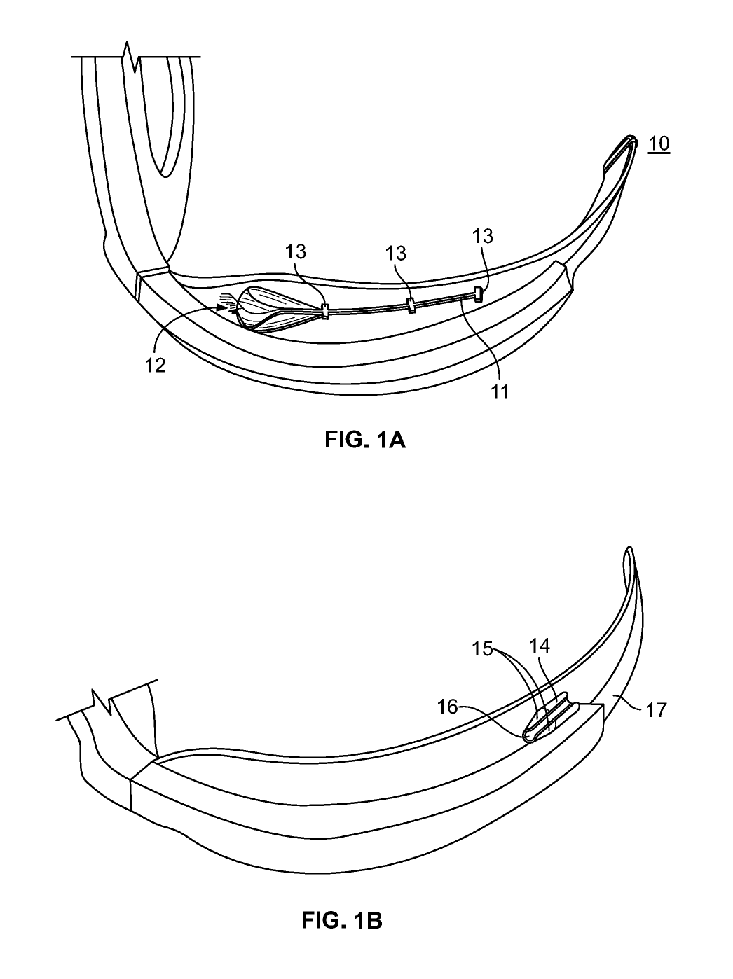

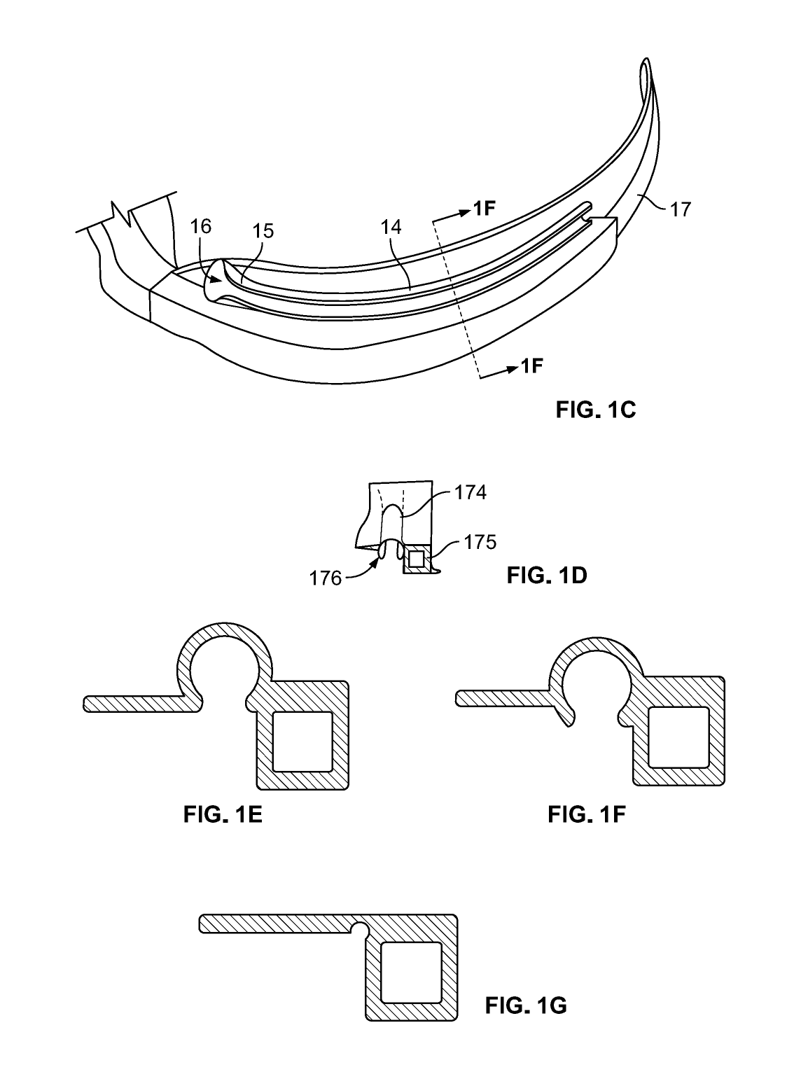

The present disclosure provides two different types of interlock mechanisms, which are illustrated in FIGS. 1A and 1B. FIG. 1A illustrates a modified video laryngoscope blade (10) having an integrated channel (11) or guide groove (interlock guide groove) designed to direct a carrier/holder (200) to the center of the field of view of a video laryngoscope. In some embodiments, the integrated channel (11) comprises a rail or a track. In some embodiments, the channel is machined into the disposable blades that mates with the video laryngoscope. In other embodiments, the disposable blade comprising the channel is formed from a mold. In some embodiments, the channel is an additional structure which may be fixed to a surface of the blade, and may comprise the same or different materials as the blade. In some embodiments, the integrated channel comprises a plurality of grooves (e.g. linear slot or spiral groove) and with perpendicular motion free recesses (13) to facilitate anterior-posterior fine-tuning and lateral/rotational adjustment of the ETT as the curved navigational tip stylet approaches the larynx. In some embodiments, a bull's eye marker or cross hairs on the screen may be utilized to confirm an optimal image acquisition during video laryngoscopy which would be maintained for procedural success.

FIGS. 1C and 1D illustrate a modified blade showing an interlock channel and, in particular, a funnel-shaped inlet (171) to an interlock channel. FIG. 1C further illustrates a low profile medial wall of the channel (172) and a stylet release slot (173). FIG. 1D illustrates a profile of a lingual surface of the blade (174). Located adjacent to the lingual surface (174) is a pathway (175), such as a pathway for a camera or a light source. FIG. 1D also illustrates a distal interlock channel and release slot (176).

FIGS. 1E, 1F, and 1G illustrate non-limiting embodiments of cross-sections of an interlock channel. FIGS. 1E, 1F, and 1G further illustrate the varying levels of embedding into the blade. FIG. 1E illustrates the maximum modification of the existing blade but retains an unimpeded view and access for regular use of the video laryngoscope without a specialized coaxial stylet. It is believed that, even with dimensions of less than about 5cm in length and having a diameter of about 5mm, the interlock channel profile protrusion onto the lingual surface should not impeded tongue displacement or diminish the laryngoscopic view achieved. FIG. 1F represents an embodiment having a compromised arrangement where there is believed to be some encroachment of the line of sight by the interlock channel during routine use. FIG. 1G illustrates the minimal modification that would be required if an ETT carrier/holder is utilized and it is secured via interlock tabs into this low profile unobtrusive groove for coaxial stylet system use, while simultaneously retaining regular operational blade characteristics when not utilized.

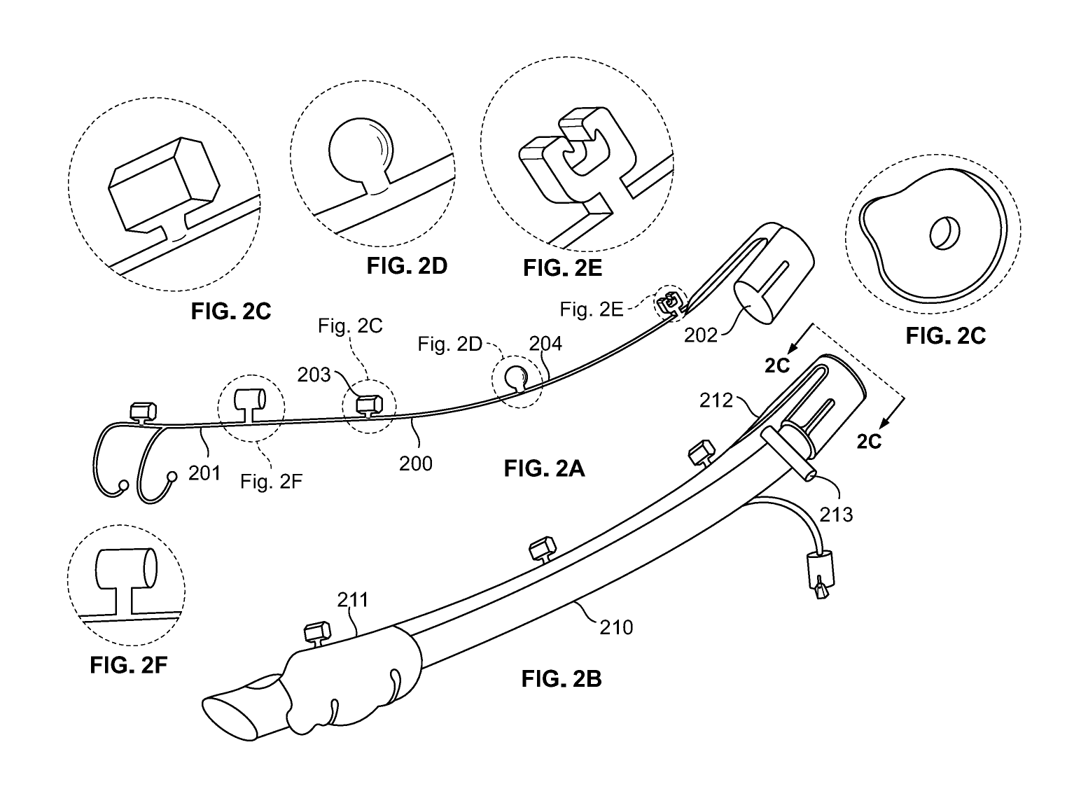

In embodiments comprising a carrier holder interlock device having a recess member (e.g. a motion free recess member), the complementary squared cylindrical interlock tabs (203) of an ETT/DLT carrier (see FIG. 2A) would simultaneously enter complete cylindrical recesses of the interlock modification of the video laryngoscope, negating rotational control of the interlock tabs while the ETT/DLT carrier remains securely in place. The recesses (13) could be within the axis of the main interlock channel with “squared” (flattened sides) rotational control along the entire channel except at the level of the recesses where a complete cylindrical form allows rotary motion if the neck of the tab is simultaneously in a perpendicular groove to allow this rotational motion. Multiple other complementary shapes of the interlock channel and the interlock tabs could be designed to accommodate this similar functional requirement (e.g. a spherical head of an interlock tab on a neck of infinite cross-sectional shapes within a sufficiently deep complementarily shaped interlock channel would not be free to rotate until it is within the motion free recess of a perpendicular channel as previously described). Again, with reference to FIG. 1A, a flared, tapered inlet (12) to the interlock guide groove is believed to facilitate easy and/or efficient mounting of the ETT/DLT carrier holder. The skilled artisan would be able to retrofit existing video laryngoscope blade profiles with the new elements of the present disclosure, subject to any design limitations in the existing laryngoscopes.

In some embodiments of interlocking the ETT/DLT delivery system with the video laryngoscope, the carrier/holder interlock mechanism is superseded by a direct coaxial stylet conduit (“DCSC”) (17), such as illustrated in FIG. 1B. This embodiment first deploys the coaxial stylet (17), with the proximally mounted ETT/DLT following afterwards. In some embodiments, the DCSC is a machined, circumferentially incomplete, cylindrical channel with a linear inner stylet release slot, variably embedded in the substance of the blade and of a variable length determined by the need for incidental emergency use as opposed to routine (every intubation attempt) use. In other embodiments, the DCSC is fixed to a surface of the blade and may be comprised of the same material or a different material than the blade.

One aspect of this design is that the inner diameter of the DCSC is of a size that allows free passage of the outer stylet of the coaxial stylet, and that entry into the DCSC is only possible at its proximal end. The inner stylet release slot (14) along the underside of the length of the DCSC allows controllable (or easy) egress of the inner stylet if it is not sheathed by the outer stylet but is critically not large enough to allow accidental dislodgment of the outer stylet. In some embodiments, the proximal opening (16) (e.g. an outer stylet entry opening) of the DCSC is tapered and rounded in a short segment type of conduit. Here, only a distal short segment controlling the coaxial stylets distal portion is required as the design of this particular embodiment is intended for use during every intubation attempt.

Once the airway is entered by the inner stylet (323), the outer stylet (324) is first retracted to disengage from the DCSC (17). It is believed that this should free the inner stylet (323) from the DCSC (17) as it is now unsheathed from the outer stylet, additionally by advancing the ETT/DLT toward the DCSC (17) the close tolerance of the inner diameter of the DCSC to the outer diameter of the outer stylet (324), as it is minimally displaced off center by the ETT/DLT, will ensure no reengagement of the outer stylet in the DCSC (17) thereafter. The tapered rounded edges of the release slot (14) may, in some embodiments, act as guide rails (15) (e.g. tapered rails) for the outer stylet if in contact with the outer surface of the DCSC. In some embodiments, the outer stylet (324) travels over the inner stylet into the trachea forming the sufficient structural support for ETT/DLT deployment over a transition free zone as described herein.

In some embodiments of a long segment-type DC SC, the proximal portion of the DCSC extends to the proximal end of the blade where it has a funnel shaped, flared segment for quick coaxial stylet installation in an emergency. The distal extent of the DCSC relative to the blade is determined by design requirements of the blade and system optics that optimizes the reliable targeting of the laryngeal introitus by the coaxial stylet as described elsewhere in this document.

In another embodiment of the DCSC (17), an appropriately larger longitudinal release slot (14) allows the outer stylet of the coaxial stylet to be controllably released from a DCSC (17) with a slightly enlarged inner diameter. In some embodiments, and as illustrated in FIG. 5B, the coaxial stylet is retained in the DCSC by an appropriate length segment of the outer stylet which is enlarged in a fusiform or cylindrical manner (500), by integrated longitudinal ridges (510), multiple circumferential rings (540), or an inter nodular low profile inflatable cuff (520 or 530) (cylindrical shape at low volumes (520), fusiform at higher volumes (530)). In this arrangement, the outer stylet is advanced over the deployed inner stylet until the enlarged segment is free of the distal opening of the DCSC, at this point the outer stylet is completely releasable from the DCSC via the longitudinal release slot. The dimensions of these changes are again dependent on constraints of the video laryngoscope blade and optics to which this embodiment is mated.

With reference to FIG. 2A, the ELT/DLT carrier/holder (200) comprises a body (204), an ETT cuff holder (201) at a first end, and a connector (202) at a second end. In some embodiments, the cuff holder (201) are one or more wires, e.g. two or more wires. In some embodiments, the cuff holder (201) and body (204) are integral, i.e. formed from a single piece of material (e.g. stainless steel, aluminum, plastic, etc.). In some embodiments, the cuff holder (201) comprises at least one wire having a semi-circular shape defining an opening. In some embodiment, the cuff holder comprises a single semi-circular wire. In other embodiments, the cuff holder (201) comprises two or more semi-circular wires. In some embodiments, the one or more wires of the cuff holder (201) define an opening that approximates the diameter of a standard ETT or DLT cuff in its inflated state. In some embodiments, the body of the carrier/holder (200) comprises a pre-formed arcuate shape.

The body of the ETT/DLT carrier comprises at least one interlock tab. In some embodiments, body of the ETT/DLT carrier/holder (200) comprises a plurality of interlock tabs (203). In some embodiments, the interlock tabs (203) are located on an upper surface of the body (204) of the ELT/DLT carrier/holder (200), such as illustrated in FIG. 2A.

FIG. 2B illustrates an ETT loaded (210) onto an ELT/DLT carrier/holder (200), such as the ELT/DLT carrier/holder depicted in FIG. 2A. More specifically, FIG. 2B illustrates the connector (202) of the ELT/DLT carrier releasably engaged to a first end of the ETT (210), and a second end (including a cuff (211)) of the ETT releasably engaged within the cuff holder (201) wires and a sliding coaxial segment (212) that allows longitudinal variability to fit multiple ETT lengths.

In some embodiments, the carrier/holder (200) is a formed from an inflexible partial cylindrical design or frame (plastic, wire or formed metal) that allows free movement of the ETT/DLT (210) during deployment. Once a cuff (211) is deflated, the carrier/holder (200) is then able to be retracted and removed. In some embodiments, proximal fixation is achieved through the use of a slitted proximal female connector cap (202) (see FIGS. 2A and 2B) that frictionally engages a standard ETT/DLT male connector (213). Without wishing to be bound by any particular theory, it is believed that this design also allows the system (i) to be introduced after the fact when an unexpected difficult airway is encountered (ii) to be pre-mounted for suspected difficult airway cases, and/or (ii) as an every-time method given its ease and safety.

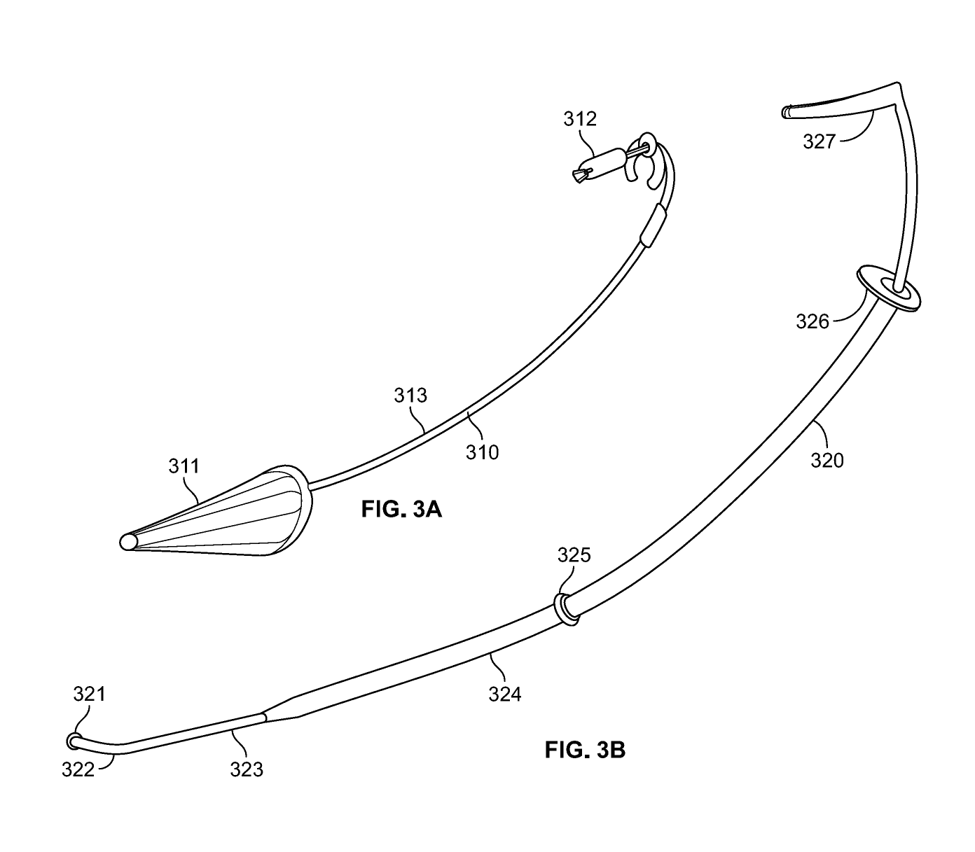

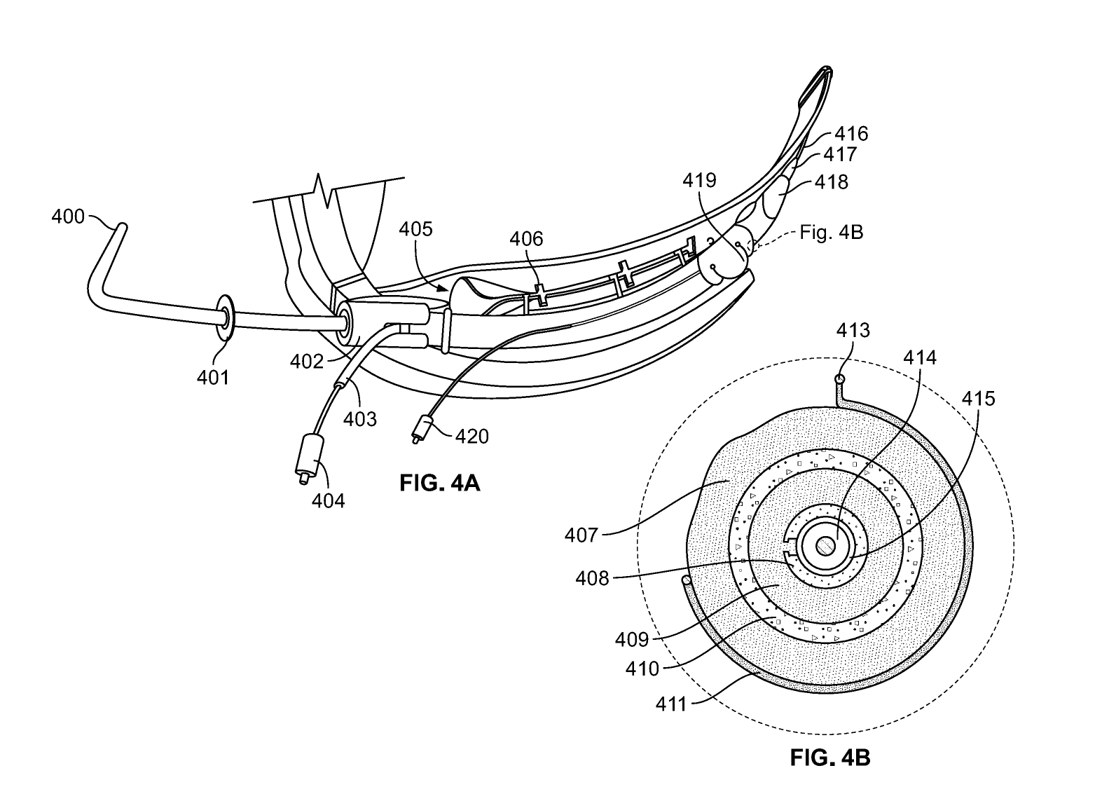

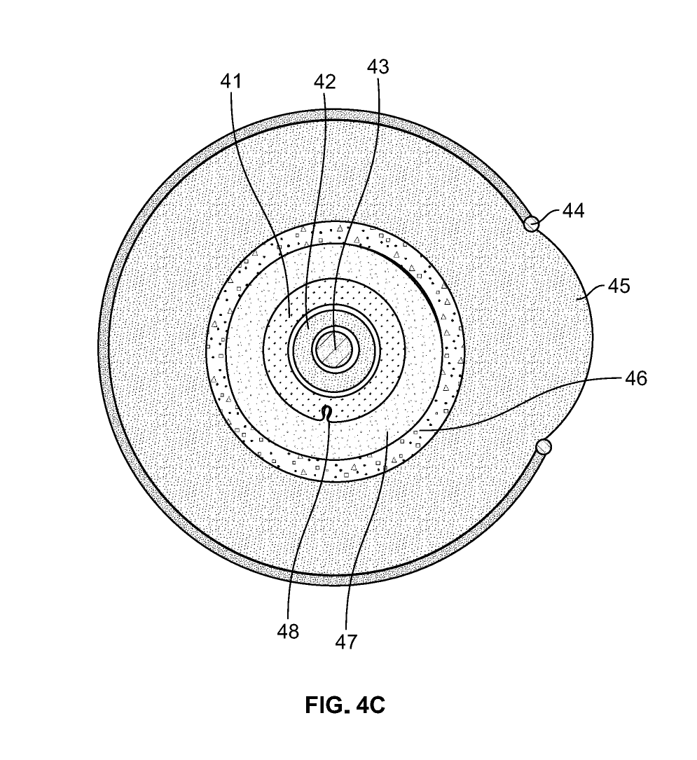

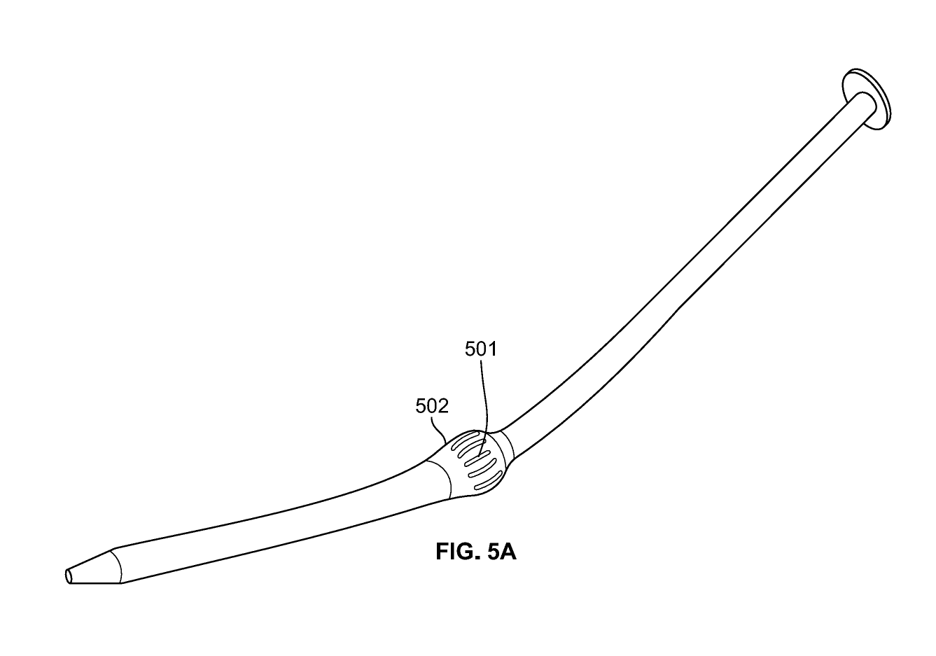

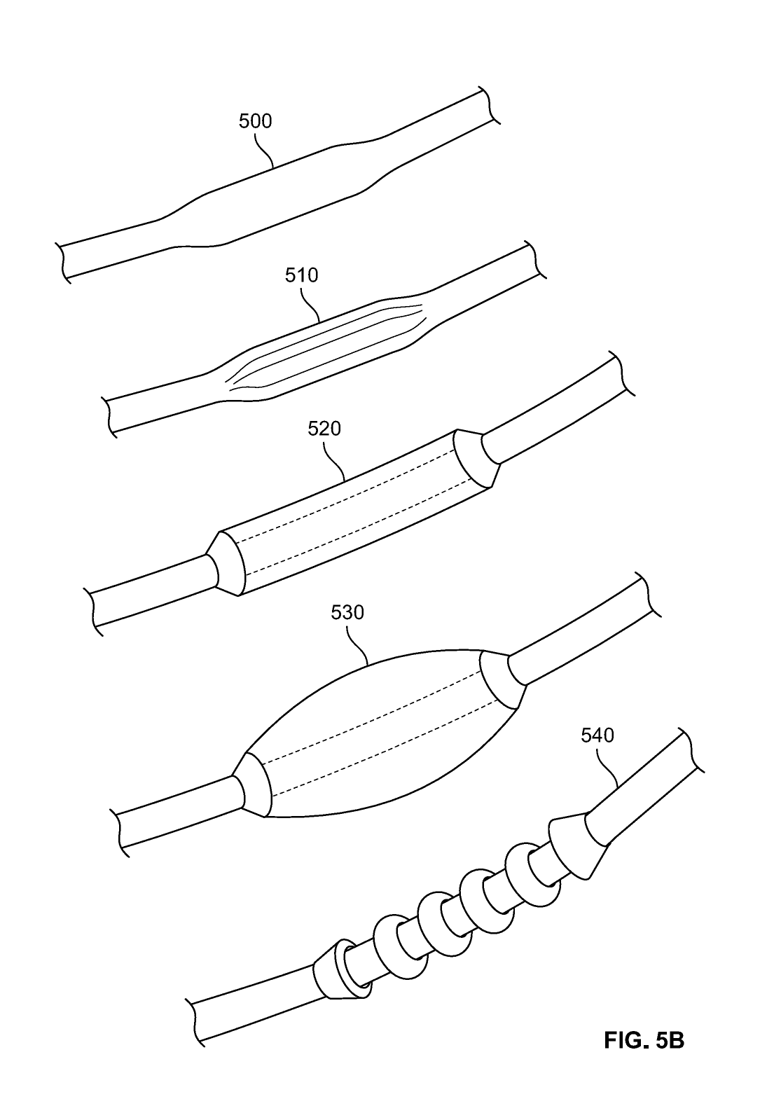

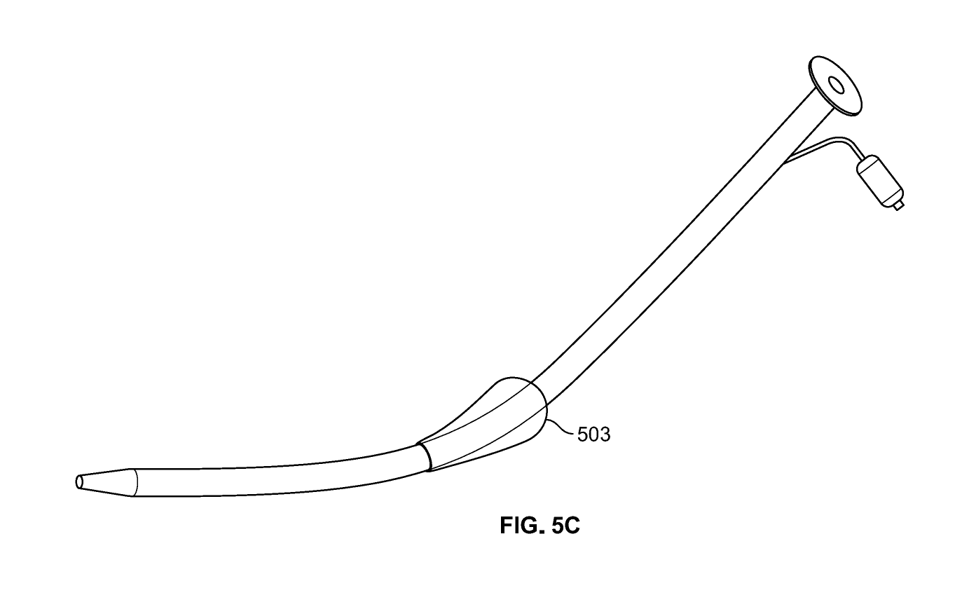

FIG. 3A illustrates a free-floating, transition free conical cuff (310). In some embodiments, the transition free conical cuff (310) comprises a pilot cuff and clip (312) at a first end, and a cuff (311) having a hollow tubular segment second at a second end. The pilot cuff (312) and the cuff (311) having a hollow tubular segment are connected by means of a low profile reinforced structural element (313). In some embodiments, the low profile reinforced structural element (313) is comprised of a material which is malleable (e.g. a metal). In some embodiments, the cuffs (311) hollow tubular structural base is linked to a wire or metal framing element that extends to the proximal end of the ETT/DLT (210) where it is fixed by the pilot clip (312) (e.g. a pilot balloon clip). This proximal end of the wire/framing element is malleable allowing it to be altered to fit varying ETT/DLT lengths.

In some embodiments, the transition free conical cuff hollow tubular structural element has a diameter greater than the outer diameter of the coaxial double stylet allowing it to slip smoothly over the coaxial stylet (320). In some embodiments, the inflatable cuff diameter dimension varies from distal to proximal to cover all ETT/DLT sizes and produces a conical transition free zone from stylet diameter to ETT/DLT diameter. In some embodiments, the cuff (311) is a low volume high pressure cuff which will allow it to maintain its substantially conical shape after inflation and its fixation to the ETT/DLT as well as creating a low profile when deflated.

FIG. 3B exemplifies a telescoping seldinger type flexible (or pliable) coaxial double stylet (320) design. In some embodiments, the coaxial double stylet has a pliable spherically tipped inner stylet (323) which deploys by telescoping into the airway. In some embodiments, the coaxial double stylet (320) has a comparatively more rigid (but still flexible) outer stylet (324) that may be advanced over the inner stylet (323) and it into the trachea. In some embodiments, the ETT/DLT is configured such that it may slide forward on the stylet interlocked with the free floating conical cuff and its smooth transition free zone facilitates railroading of ETT/DLT into the trachea. In some embodiments, there exists a curved distal navigation tip (322) at an end of the inner stylet (323), the spherical or arcuate contour of the inner stylet tip (323) has a shallow recess for the outer stylet (324) mating surface producing an atraumatic (321) ETT/DLT co-axial stylet composite unit for deployment. In some embodiments, inner stylet (323) extends from about 10 cm to about 15 cm from the proximal end of the outer stylet (324) with a steering rotational controlling tab/arm/dial (327) doubling as a stop (326). In some embodiments, the stop limits how much the device may telescope out the distal end. Without wishing to be bound by any particular theory, it is believed that the design may prevent or mitigate distal airway trauma or carinal stimulation as this becomes the limit of outer stylet and composite ETT/transition free conical cuff deployment. The use of color coded markings and physical deployment stop (326) limits will enhance the safety aspect of this design. As the inner stylet is independently deployed to its limiting tab, an integrated ring (325) of material on the outer stylet (324) having a diameter greater than the inner diameter of the conical cuff tubular segment, prevents additional independent motion of the outer stylet and thus functions as such a deployment stop limit for the outer stylet.

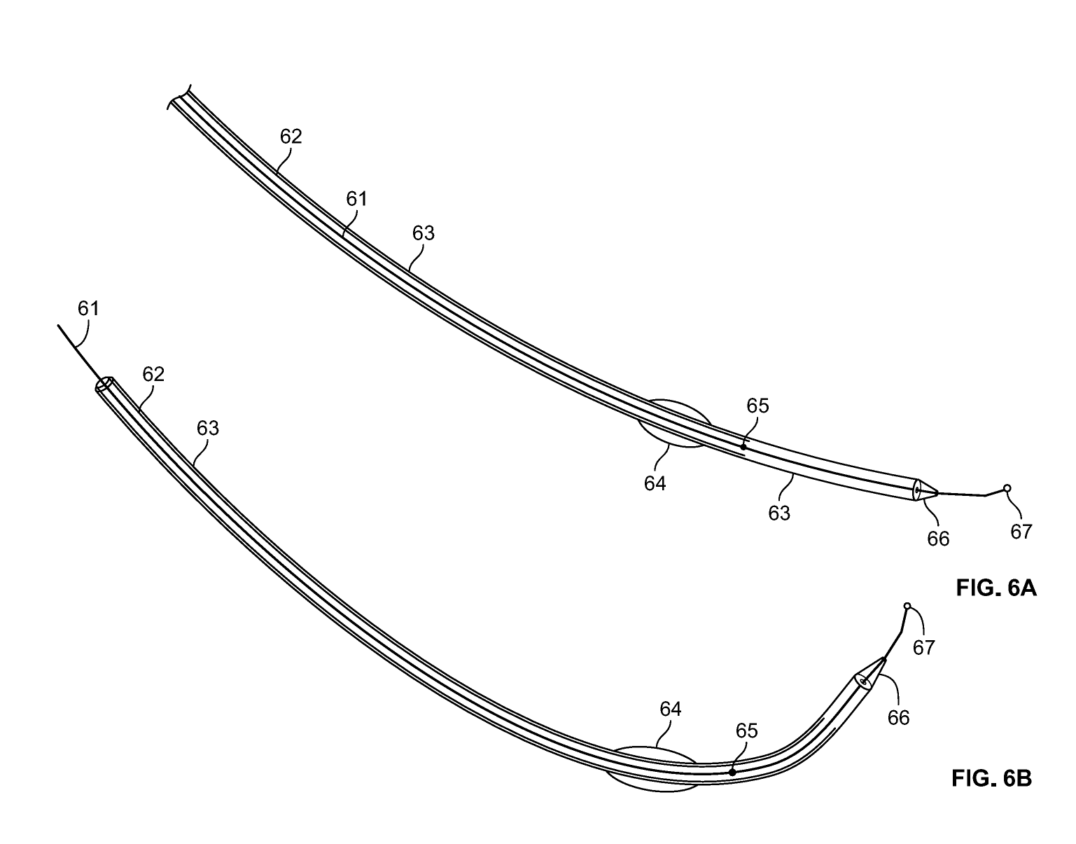

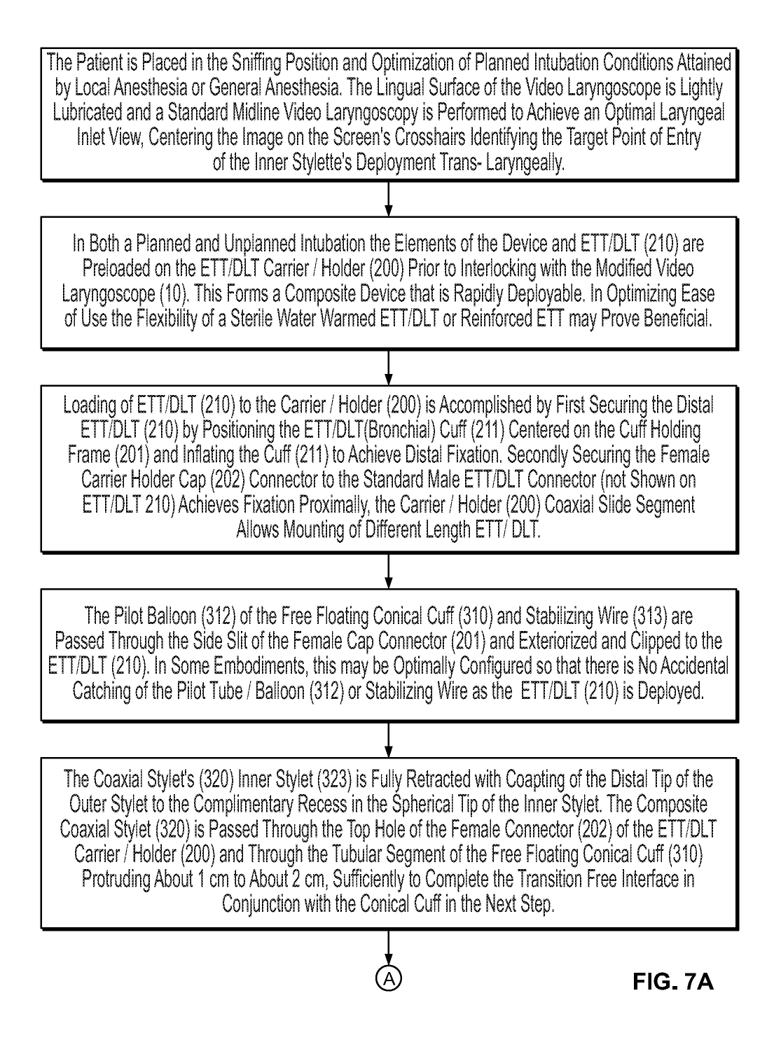

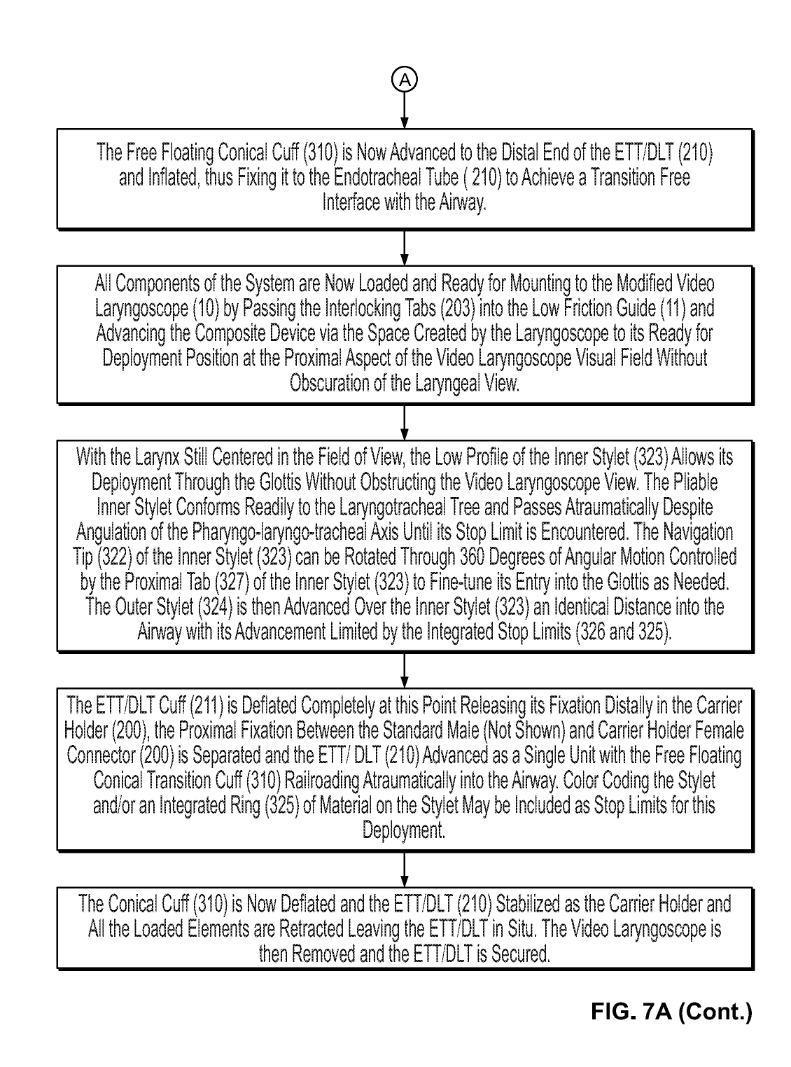

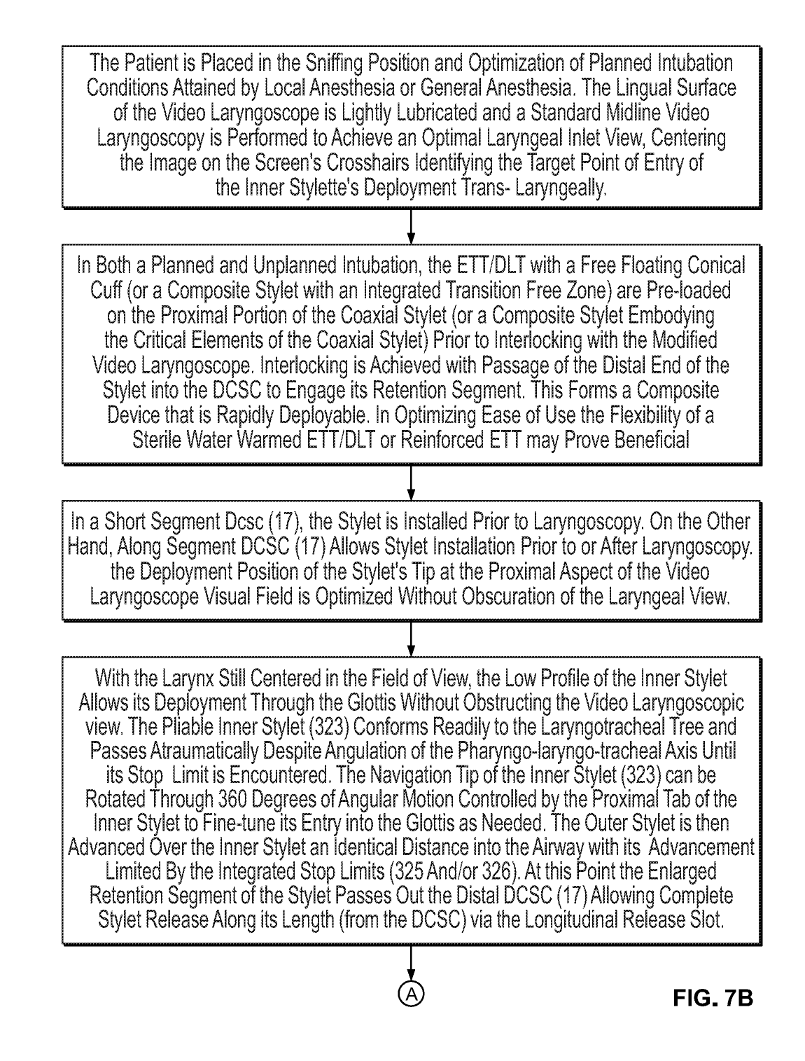

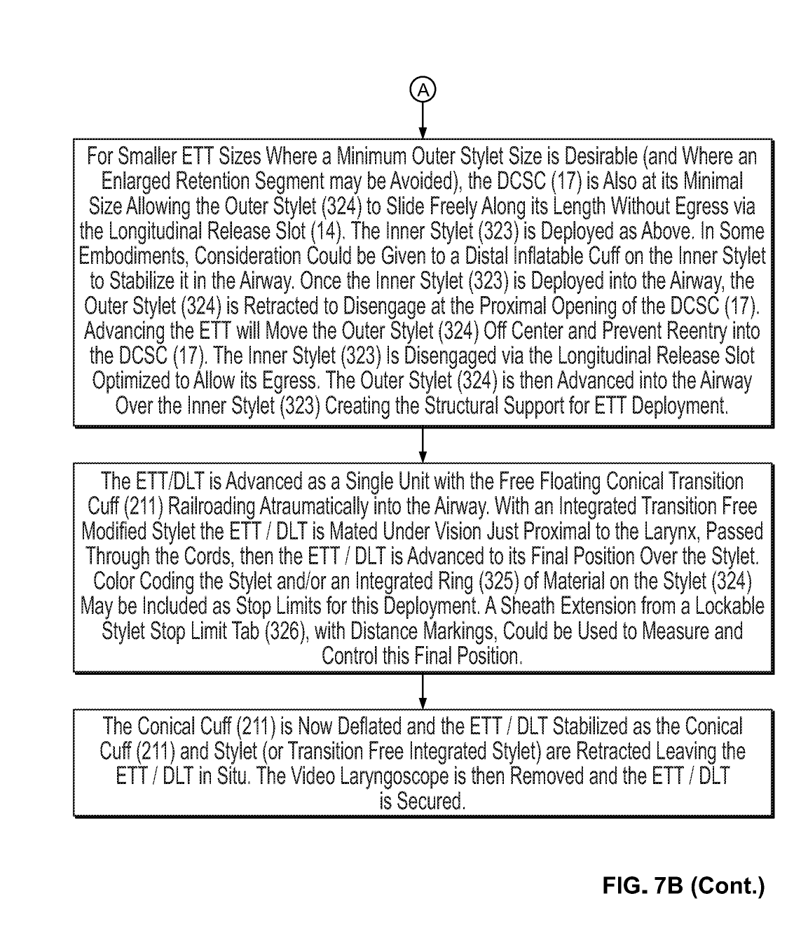

Also disclosed are methods of performing endotracheal intubation system for performing an endotracheal intubation and assembling an endotracheal intubation system (see FIG. 6).

In some embodiments, the telescoping coaxial stylet with free floating conical cuff for DLT difficult intubation is achieved via the bronchial lumen which is optimized to approach the cords, then the stylet's pliable telescoping tip traverses the cords.

In some embodiments, a separate malleable shaping stylet facilitates curving of DLT to more easily conform to the video laryngoscope shape via tracheal lumen in a modified DLT design.

In some embodiments, the free-floating transition free conical cuff can be mated with one or more of a fiber optic bronchoscope, a rigid fiber optic scope, a light wand, an ETT exchanger and/or a bougie to enhance the success of using these other intubation devices. Without wishing to be bound by any particular theory, it is believed that the free-floating design is important in that the normal manipulation of these devices is achieved with the mounted ETT/free floating conical cuff in the typically preferred most proximal location on these devices to avoid interference during their deployment or can be mounted on the device after the fact as in the case of the ETT exchanger. In the case of a flexible fiber optic scope, by reinforcing the malleable structural frame element of the free floating conical cuff the ability to manipulate the position of the ETT/DLT tip fixed to the free-floating cuff may enhance the ability to enter the laryngeal introitus. Additionally, a segmental outer diameter enlargement modification of a fiber optic bronchoscope to make it DCSC compatible (passable and controllable via the DCSC (17)) would, it is believed, facilitate the technique of combined video laryngoscope and fiber optic bronchoscope assisted intubation.

In another embodiment, is an endotracheal tube for the pediatric population, the tubes having sizes and inner diameters which are decreased comparatively to the adult embodiments disclosed herein. In some embodiments, the coaxial stylet/free floating cuff design is further modified for pediatric use. For example, the device may be modified into a unitary composite element with the inclusion of critical elements of its parent parts given endotracheal tube luminal spatial limitations (see FIGS. 5A and 5B). This would then require ETT/DLT size specific alternate stylets for these smaller sized items. With these ETT/DLT deployment over the stylet would be initiated over a stabilized stylet position as soon as the transition free zone traverses the cords. Unique to these modified stylets would be the location of the transition free point at a distance which would place the stylet tip at or just beyond the mid tracheal distance when the transition free zone has just traversed the cords.

In another embodiment, nasal intubation may be accomplished by a string loaded retrieval mechanism that unites the nasally placed endotracheal tube and the endotracheal tube carrier ETT holder or a distal extension of the video laryngoscope interlock would allow maximal advancement of the carrier holder while still secured to the video laryngoscope. This positioning of the holder would then allow easy uniting of the trans-nasally passed ETT with the carrier holder under video laryngoscopic visual guidance; the united components can then be retracted to its normal deployment position.

In another aspect of the present disclosure are kits comprising any of the components of the present disclosure. In some embodiments, the kit comprises (i) a modified laryngoscope; (ii) a ETT or DLT; (iii) means for securing the ETT or DLT to the modified laryngoscope; (iv) and a stylet. In some embodiments, the ETT/DLT is pre-packaged for sale resulting in economies of scale and speed of deployment in an unexpected emergency.

### Additional Embodiments

Additional Embodiment 1. A laryngoscope blade comprising a direct coaxial stylet conduit allowing independent sequential deployment of coaxial stylet components.

Additional Embodiment 2. The laryngoscope blade of additional embodiment 1, wherein the direct coaxial stylet conduit comprises an inner stylet release slot, at least one tapered rail, and an outer stylet entry opening.

Additional Embodiment 3. A coaxial stylet comprising inner and outer stylets, the inner stylet comprising a steering arm at a first end, and a spherical tip at a second end, and wherein the inner stylet further comprises a navigational segment.

Additional Embodiment 4. The coaxial stylet of additional embodiment 3, wherein a movable stop limit circumscribes a portion of the outer stylet.

Additional Embodiment 5. The coaxial stylet of additional embodiment 3, wherein the coaxial stylet further comprises a second stop member.

Additional Embodiment 6. The coaxial stylet of additional embodiment 3, wherein the navigational segment is curved or arcuate.

Additional Embodiment 7. A laryngoscope blade comprising a channel, a plurality of recesses disposed along the length of the channel, the recesses having a width which is greater than a width of the channel, and wherein one end of the channel comprises an interlock groove inlet.

Additional Embodiment 8. The laryngoscope blade of additional embodiment 7, wherein the channel and recesses are machined into a laryngoscope blade.

Additional Embodiment 9. An ETT/DLT carrier/holder comprising a connector cap, one or more arcuate cuff holder wires, and a sliding coaxial segment.

Additional Embodiment 10. A kit comprising: (i) the laryngoscope blade of any additional embodiment 3 to 6; and (ii) the coaxial stylet of any of additional embodiments 21-24.

Additional Embodiment 11. The kit of additional embodiment 10, further comprising an endotracheal tube.

Additional Embodiment 12. The kit of additional embodiment 10, further comprising the ETT/DLT carrier/holder of additional embodiment 27.

Additional Embodiment 13. The kit of any of additional embodiment 10 to 12, further comprising instructions for intubating a patient.

Additional Embodiment 14. The kit of additional embodiment 10, wherein the coaxial stylet is sized for a pediatric patient.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. An endotracheal intubation system for performing an endotracheal intubation, comprising:
a laryngoscope including a laryngoscope blade, the laryngoscope including a channel having a plurality of recesses configured to releasably engage an ETT/DLT carrier/holder;
an endotracheal tube releasably secured to the ETT/DLT carrier/holder by fixation means; and
a coaxial stylet for the endotracheal tube, the coaxial stylet including inner and outer stylets and an inner stylet steering arm.

2. The endotracheal intubation system of claim 1, wherein the fixation means comprise a connector cap, cuff holder wires, and a sliding coaxial segment that allows longitudinal variability to fit multiple ETT lengths.

3. The endotracheal intubation system of claim 1, further comprising a transition free conical cuff.

4. The endotracheal intubation system of claim 4, wherein the coaxial stylet is configured to pass through a hollow tubular segment of the transition free conical cuff.

5. The endotracheal intubation system of claim 1, wherein the coaxial stylet comprises at least one means for restricting advancement of the inner stylet.

6. The endotracheal intubation system of claim 5, wherein the at least one means for restricting advancement of the inner stylet is a stop limit disc coupled to an end of the outer stylet.

7. The endotracheal intubation system of claim 5, further comprising at least one means for restricting advancement of the outer stylet.

8. The endotracheal intubation system of claim 1, wherein the ETT/DLT carrier/holder is reliably engaged to the recesses of the laryngoscope by a plurality of interlock tabs.

9. The endotracheal intubation system of claim 1, wherein the inner stylet comprises an arcuate or curved navigational end, the arcuate or curved navigational end opposite the inner stylet steering arm.

10. The endotracheal intubation system of claim 1, wherein the inner stylet comprises an atraumatic spherical tip.

11. An endotracheal intubation system for performing an endotracheal intubation, comprising:
a laryngoscope including a laryngoscope blade, the laryngoscope including a direct coaxial stylet conduit configured to releasably engage a coaxial stylet; and a coaxial stylet for an endotracheal tube, the coaxial stylet including inner and outer stylets and an inner stylet steering arm.

12. The endotracheal intubation system of claim 11, herein the coaxial stylet includes inner and outer stylets and an inner stylet steering arm.

13. The endotracheal intubation system of claim 11, wherein the direct coaxial stylet conduit permits independent sequential deployment of the coaxial stylet.

14. The endotracheal intubation system of claim 11, wherein the direct coaxial stylet conduit comprises an inner stylet release slot, at least one tapered rail, and an outer stylet entry opening.

15. A method of deploying an endotracheal tube into a patient's mouth between the patient's vocal cords into the patient's larynx, and into the patient's trachea for opening the patient's airway, the method comprising deploying and positioning an endotracheal tube using the system of any of claims 1 to 14.

16. A laryngoscope blade comprising a direct coaxial stylet conduit configured to receive a region of an outer stylet that is circumferentially larger than an adjacent region, the region that is circumferentially larger comprising at least one retention feature.

17. The laryngoscope blade of claim 16, wherein the retention feature is selected from the group consisting of integrated longitudinal ridges, circumferential rings, and an inter nodular low profile inflatable cuff.

18. The laryngoscope blade of claim 16, further comprising a longitudinal release slot which permits disengagement of the outer stylet along a portion which is not enlarged.
